(19) United States
(12) Patent Application Publication
(10) Pub. No.: US 2008/0031783 A1
(43) Pub. Date: Feb. 7, 2008

(54) Photocatalytic fabric

(76) Inventors: Daniel J. Briggs, Lubbock, TX (US); Carl F. Varnerin, Norton, MA (US)

Correspondence Address: AARON R. CLEMENTS/HURLEY, REYES & GUINN, 1805-13TH ST., LUBBOCK, TX, 79401, US

(21) Appl. No.: 11/099,230

(22) Filed: Apr. 2, 2005

(51) Int. Cl. A61L 9/20; B32B 5/02; D03D 9/00

(52) U.S. Cl. 422/121; 442/1; 442/43

(57) ABSTRACT

A multipurpose air sterilization machine is disclosed comprising an enclosure holding a pre-filter, an ultraviolet light chamber with a plurality of ultraviolet lights, one or more photocatalytic oxidizing scrims, and a blower. Air is pulled through the machine by the blower, and passes first through the pre-filter, then the first scrim, then one or more ultraviolet light chambers defined by the plurality of scrims, and is ultimately expelled from the rear of the machine after passing through the blower. The scrims provide for enhanced ultraviolet decomposition of organic compounds, including bacteria, mold spores, viruses, and other organisms, and comprises a substrate coated with a photocatalytic material such as TiO₂ adhered to the surface with an ultraviolet-transparent organic polymer.

### REFERENCE TO PRIOR APPLICATIONS

Reference is hereby made to Provisional application No. 60/424,270, filed Apr. 2, 2004, from which priority is claimed pursuant to 35 U.S.C. § 120.

### STATEMENT REGARDING FEDERALLY SPONSORED RESEARCH

Not Applicable.

### REFERENCE TO “MICROFICHE APPENDIX”

Not Applicable.

### BACKGROUND OF THE INVENTION

This Invention relates to devices for purifying and sterilizing air using a photocatalytic scrim formed by coating a substrate with titanium dioxide (TiO₂). Purification and sterilization of air is beneficial for a number of applications from the medical field to home/consumer use. In the medical field, for instance, hospitals rely on sterilization to prevent infection of patients undergoing surgery. Given that a hospital, by its nature, has a large concentration of sick persons, air sterilization is desirable to minimize the risks of diseases being communicated to noninfected persons, both patients and staff. In commercial use, air purification technologies are critical in a number of applications, including the remediation of indoor air pollutants, especially including organic solvents to which exposure has been determined to be harmful. Additionally, air purification and sterilization machines have applications in home use, particularly in the context of efforts to remediate water damage to property. In such situations, the damp conditions caused by water damage can result in the explosive growth of both bacteria and molds, with the entire building being contaminated by airborne bacteria and mold spores. In fact, mold spore contamination resulting from water damage, particularly with respect to the mold Stacybotrys chartarum, has resulted in a current crisis among homeowners and insurers. Insurance companies are currently losing billions of dollars on mold claims, and are either increasing insurance premiums drastically or ceasing to do business in many states.

Photocatalytic oxidation of contaminants is a highly desirable and rapidly developing field which provides extremely cost-effective solutions to the problems of removing airborne contaminants. Photocatalytic oxidation (PCO) results from using a catalyst, typically titanium dioxide (TiO₂) in the presence of ultraviolet light (particularly UV-C band light) to convert contaminants containing organic materials into principally carbon dioxide and water. Typically, the titanium dioxide is supported by coating onto a substrate, typically metal or fiberglass. See, e.g., Kobayashi et al., U.S. Pat. No. 6,368,668; Kimura et al., U.S. Pat. No. 6,407,033; Chopin et al., U.S. Pat. No. 6,362,121; Ito et al., U.S. Pat. No. 6,683,023; Nishii et al., U.S. Pat. No. 6,820,752; Scott et al., U.S. Pat. No. 6,508,367; Yu, U.S. Pat. No. 6,803,077; Gonzalez-Martin et al., U.S. Pat. No. 6,136,186; Robertson et al., U.S. Pat. No. 4,892,712; Robertson et al., U.S. Pat. No. 5,032,241; Goswami, U.S. Pat. 5,835,840.

Existing machines for sterilization and decontamination of air include deodorizers, chemical- and charcoal-based air scrubbers, ozone producers, simple filtration systems, ionization apparatus, and exhaust fans. These existing systems are typically dependent on existing environmental heating/air conditioning systems to provide air flow in order to treat large, multi-room volumes of air. None of these devices is capable of filtering and sterilizing air as well as providing an air stream for drying a large volume. One embodiment of the instant device provides a single integrated device for the rapid sterilization of air contaminated by a wide variety of organisms, including bacteria and mold spores, as well as a wide variety of organic compounds, and provides its own airflow capable of drying an area or volume that has sustained water damage. This rapid sterilization will substantially reduce the costs associated with decontaminating a premises that has sustained water damage, resulting in substantial savings to both property owners and insurance companies. Additionally, while the apparatus may be built as an independent unit, it may also be integrated into existing heating/air conditioning systems to provide for ongoing air purification in applications requiring ongoing remediation. Since the unit is built as a single machine, the instant invention will also substantially increase the efficiency and availability of decontamination/restoration processes.

The instant device attains its high efficiency from the use of a photocatalytic material incorporated into the device in the form of titanium dioxide coated onto a substrate material, which substrate is introduced into the air stream and illuminated with ultraviolet light. While there are many different materials that may be used for the substrate, including metal plate, metal wire, ceramic substrates, and glass substrates (including fiberglass), the preferred embodiment of the instant invention includes a scrim formed from a woven fiberglass fabric. The weave of the fiberglass allows the fabric to be folded into complex shapes as required, including pleats or rolls. Problems associated with loss of the photocatalyst from the surface of the substrate material are alleviated through a coating process including the use of an organic polymer binder which is substantially transparent to ultraviolet light.

### SUMMARY OF THE INVENTION

The instant Invention is designed to be a stand-alone, independent air sterilizer. The machine operates in a manner similar to existing filtration systems, and in fact incorporates some aspects of existing filtration systems, but includes some important differences. The essential components of the overall apparatus are an enclosure containing an air intake system, a pre-filter system, an ultraviolet light chamber, and a photocatalytic oxidizer. The air intake system comprises a blower and blower motor which pulls air into the machine, causing the air to pass through a pre-filter. This pre-filter can be selected from a number of commercially available filters. This pre-filter removes a substantial portion of the existing particulate from the air stream through the machine. After the pre-filter, air enters an ultraviolet light chamber. Ultraviolet light has been known to cause the breakdown of organic compounds and additionally is harmful to bacteria. As the air flows into and out of the ultraviolet light chamber, it passes through a pair of photocatalytic oxidizers, one placed at the entrance to the ultraviolet light chamber and the other placed at the exit.

The photocatalytic oxidizer, or “scrim,” preferably comprises a mesh substrate coated with titanium dioxide (TiO₂) bound to the substrate by use of an organic binder, preferably an organic polymer which is substantially transparent to ultraviolet light. This scrim forms at least one wall of the ultraviolet light chamber and is bathed in ultraviolet light, particularly of the wavelengths referred to in the art as ‘ultraviolet C’ or ‘UV-C,’ more particularly of wavelengths of approximately 254 nanometer range. Additionally, by the inclusion of multiple scrims placed perpendicular to the air stream, a plurality of chambers may be formed, greatly enhancing the decontamination process. The titanium dioxide coating on the wire mesh enhances the ability of the ultraviolet light utilized in the apparatus to destroy organic compounds. As organic compounds (including those present in the cell walls of bacteria, mold spores, viruses, and other such contaminants) are pulled in an airstream through the photocatalytic oxidizer, the titanium dioxide, in conduction with ultraviolet light, breaks them down into carbon dioxide and water. The resultant carbon dioxide and water products are then outgassed with the airflow as it passes through the photocatalytic oxidizer. Because these products are natural components of the atmosphere, they are noncontaminants, and since the photocatalytic oxidizer converts organic and biological contaminants into these products, the invention is self-cleaning and needs substantially less maintenance than existing systems. The fan speed of the machine may be varied depending on the amounts and types of material to be removed from the air stream. The device is scalable from small units suitable for individual rooms, apartments, or houses, up to large units suitable for decontaminating and sterilizing air in industrial settings.

As noted earlier, the scrim in the preferred embodiment of the invention is formed by using a woven fiberglass fabric substrate and coating it with a mixture of titanium dioxide and an organic binder. The woven fiberglass fabric includes spaces between the glass fibers to allow passage of a contaminated air stream through the fabric. The fiberglass scrim is coated with a mixture of essentially pure anatase TiO₂ and an organic binder which is transparent or translucent to UV-C, which is preferably a methylated melamine resin or an acrylic latex comprising approximately 60 percent of a styrene, butyl acrylate, acrylonitrile, and methacrylic acid polymer, about 40 percent by weight, water, about 0.2 percent by weight, ammonia, and less than 0.001 percent by weight, acrylonitrile. While the exact manner of manufacture is not disclosed herein, the preferred finished scrim includes from 0.5 percent to 20 percent TiO₂ by weight, more preferably from 2 percent to 5 percent. The TiO₂ used is preferably anatase crystals of a size between 20 nm and 15,000 nm, but is most preferably of 40 nm sized crystals exhibiting equiaxial shape. Furthermore, the finished product also includes from 0.5 percent to 20 percent organic binder by weight, more preferably from 2 percent to 5 percent. The preferred substrate makes up the balance of the weight of the scrim, and may be made from a woven and/or nonwoven material, or combination thereof, but is preferably made of a woven material with openings between fibers from 0.020 to 0.100 inches. The material comprising the scrim may be organic or inorganic, but is preferably inorganic, and more preferably fiberglass. The mixture of TiO₂ and organic binder is applied to the scrim in such a manner that the openings between fibers is not occluded. By this arrangement, a fluid may pass through the scrim, and the surface area of TiO₂ exposed to the fluid is greatly increased.

By way of additional disclosure, the following material is incorporated from Provisional application No. 60/424,270:

### DESCRIPTION OF THE DRAWINGS

FIG. 1 shows an oblique cutaway view of the apparatus showing the relative positions of the medium grade prefilter, two photocatalytic scrims, and blower.

FIG. 2 shows an oblique cutaway view of the apparatus with the filters removed and showing the prefilter area, photocatalytic oxidation chamber, and blower.

FIG. 3 shows a detailed view of the surface of the photocatalytic scrim.

FIG. 4 is a cross-sectional view of the apparatus, showing particulate flow through the apparatus and removal of contaminant at each stage of filtration and purification.

FIG. 5 is a cross-sectional view of the prefilter and photocatalytic chamber, demonstrating ultraviolet irradiation of the photocatalytic scrims.

FIG. 6 is a cross-sectional view of the prefilter and photocatalytic chamber as installed in a duct of an existing environmental conditioning system.

FIG. 7 is a cross-sectional enlarged view of the weave pattern of the photocatalytic scrim.

FIG. 8 is an oblique enlarged view of a large section of the photocatalytic scrim.

### DETAILED DESCRIPTION OF THE INVENTION

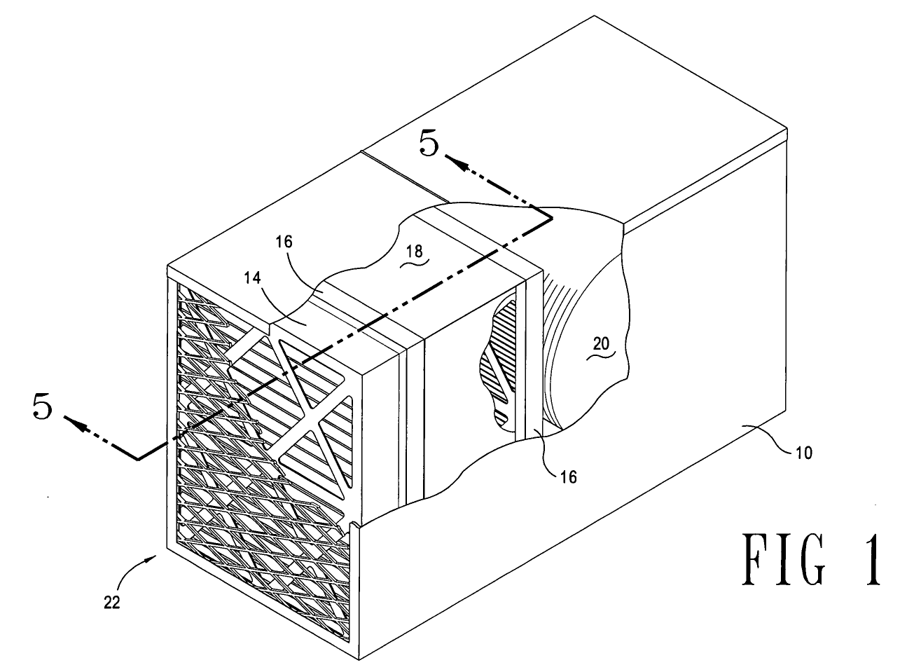

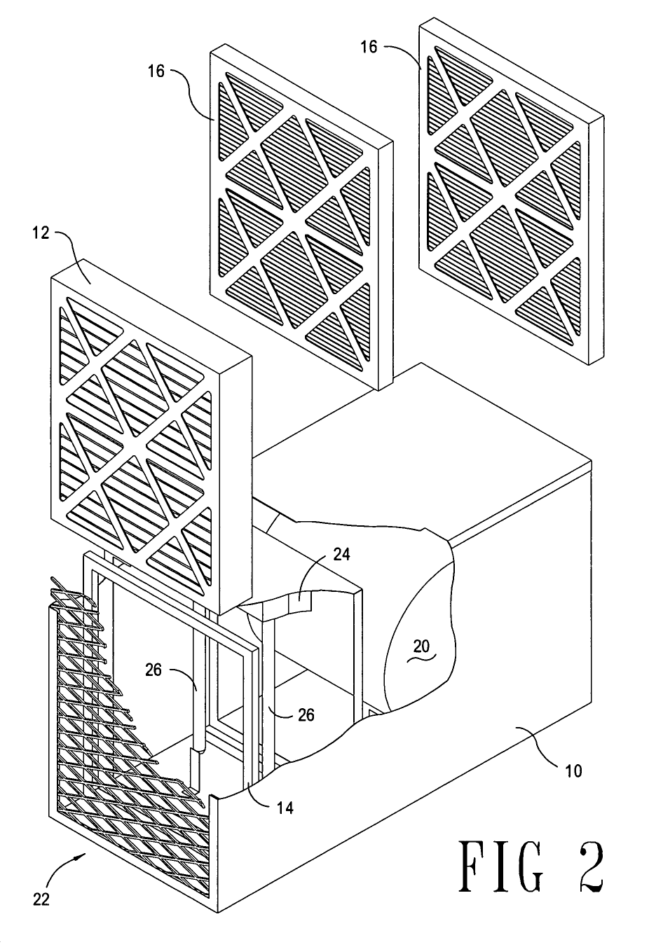

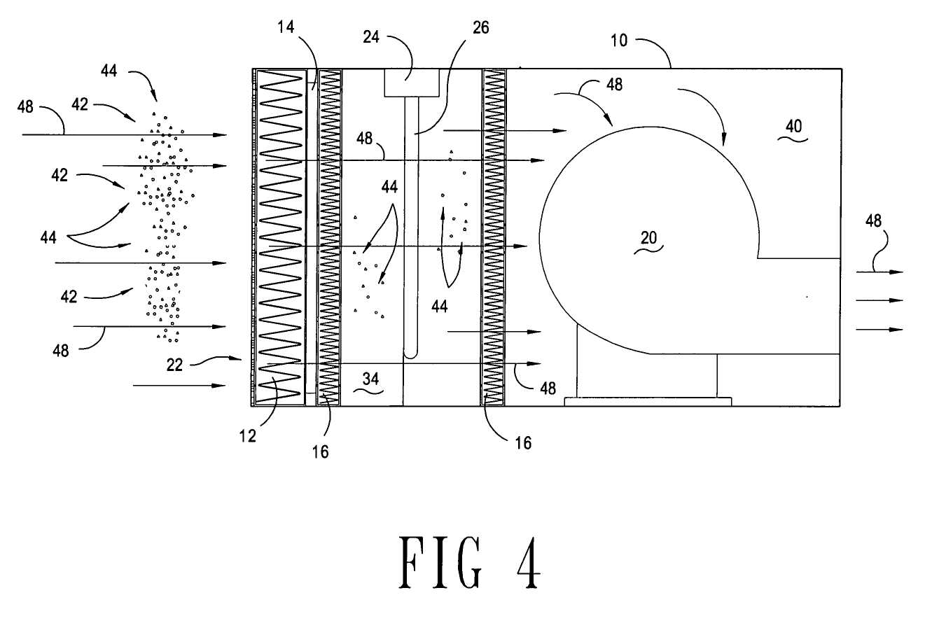

Referring to FIGS. 1, 2, and 4, the instant Invention comprises an enclosure (unit housing) 10 having a top, two sides, a bottom, as well as a forward opening (air inlet) 22 and a rear exhaust (air duct) 48. The enclosure 10 includes a conventional air filter 12, an ultraviolet photocatalytic chamber 34, and means for moving a fluid stream through the enclosure 20. It will be understood that while the instant invention is preferably used to purify a fluid stream which is gaseous in nature, the same principles may apply to removal of contaminants from a liquid fluid stream. The forward opening 22 allows a gas containing contaminant particulates 42 and microscopic particulates 44 to pass into the apparatus by flowing initially through the air filter 12. The air filter 12 is housed between the forward opening 22 and an air sealant 14, which is essentially a flange running around the interior diameter of the enclosure 10. The contaminant particulates 42 are macroscopic contaminants and are trapped by the air filter 12. The contaminated gas stream 48 then passes anterior through an initial photocatalytic scrim 16 and into a photocatalytic chamber 34. In this region, one or more ultraviolet light bulbs 26 powered through ultraviolet power ballasts 24 are positioned to emit ultraviolet light 36 within the chamber 34, thereby activating the photocatalyst 54. Microscopic contaminants 44 and molecular contaminants are decomposed into principally carbon dioxide and water vapor as the contaminated gas stream 48 passes through the scrims 16. The gas stream 48 then passes into a purified air chamber 40 and is vented to the rear of the apparatus through an outlet air duct 46 by means 20 for moving the gas stream 48, which will be understood to preferably be an induction fan.

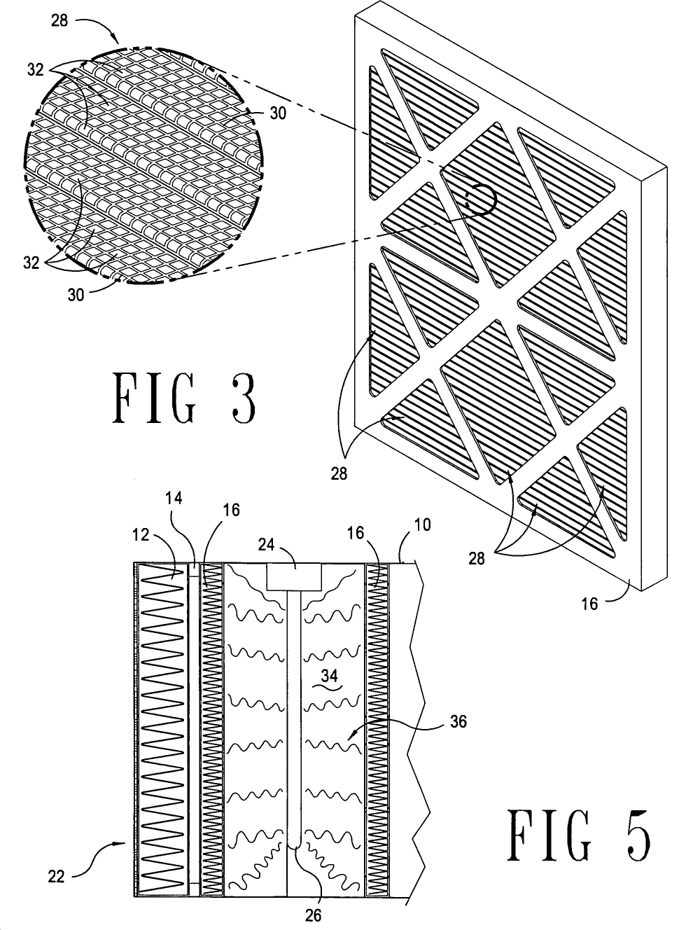

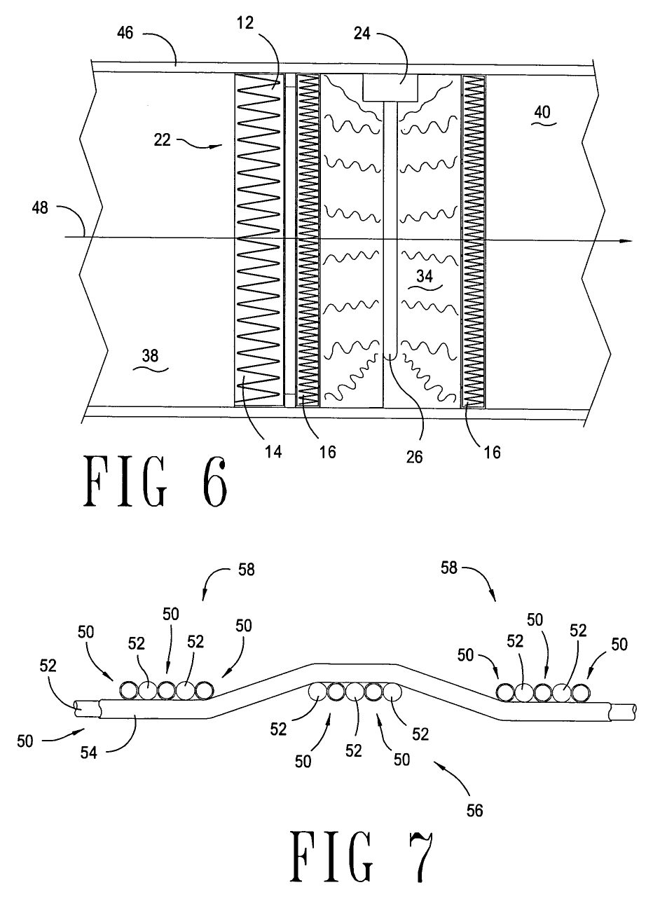

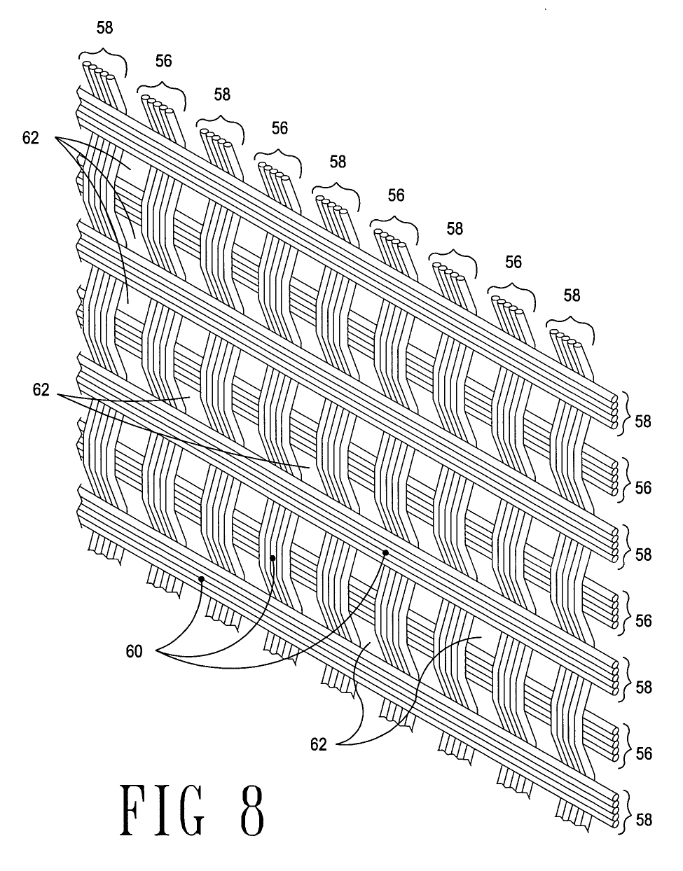

Referring to FIG. 6, it will be understood that the same arrangement may be made within an existing ventilation duct 46, utilizing an existing heating, ventilation, and air conditioning system to move the gas stream 48 through the air filter 12, scrims 16, and photocatalytic chamber(s) 34. Referring to FIGS. 3, 7, and 8, it will be understood that the preferred material for making the scrim 16 is a woven material with a plurality of opening. FIGS. 3, 7, and 8 are substantially enlarged to show details not otherwise viewable to the naked eye. Referring to FIG. 3, it will be understood that the material on which the photocatalyst 54 is coated is a flexible material which may be folded or pleated to provide increased surface area and photocatalyst 54 on which the photocatalytic reaction may take place. Furthermore, referring to FIGS. 7 and 8, it will be understood that the material from which the scrim is made preferably comprises strands of fiberglass 50 and 52 with a plurality of openings 62 formed in the weave through which the gas stream 48 may pass.

On the scrim 16 comprising fiberglass strands 50 and 52, the photocatalyst is adhered to the surface of the scrim 16 by an organic binder, preferably an organic polymer that is transparent (or at least translucent) to ultraviolet light. As noted previously, the scrim 16 comprises between 0.5 and 20 percent TiO₂ and between 0.5 and 20 percent organic binder, with the fiberglass strands 50 and 52 comprising the remainder of the weight of the scrim. Preferably, the percentages of TiO₂ and organic binder present are each between 2 and 5 percent of the total weight of the scrim 16. The organic binder keeps the anatase crystalline TiO₂ from flaking or otherwise coming loose from the surface of the fiberglass strands 50 and 52. Also preferably, the organic binder comprises a methylated melamine resin or an acrylic latex comprising approximately 60 percent of a styrene, butyl acrylate, acrylonitrile, and methacrylic acid polymer, about 40 percent by weight, water, about 0.2 percent by weight, ammonia, and less than 0.001 percent by weight, acrylonitrile. As noted, the preferred photocatalyst is anatase crystalline TiO₂ of particulate size between 20 nm and 15,000 nm, with a more preferred particulate size of approximately 40 nm.

While the instant description only includes one prefilter, it will be readily understood that more than one prefilter may be provided to achieve maximum removal of large particulate matter, especially with particulate sizes greater than one micron. It will further be understood that while the instant description only includes one ultraviolet chamber, by adding successive scrims to the apparatus, a series of ultraviolet chambers may be defined, within which the decontamination process may be enhanced. Those of ordinary skill in the art will understand the various parts of the apparatus.

### Catalogue of Elements

10 Unit Housing
12 Pre-filter
14 Air sealant
16 Scrim
18 UVGI Housing
20 Means for Air Circulation
22 Air Inlet
24 UVGI Power Ballast
26 UVGI Bulb
28 Catalytic Screen
30 Treated Catalyst Surface (TiO₂)
32 Air Flow Aperture
34 UVGI Chamber
36 UVGI Light
38 Contaminated Gas
40 Clean Gas Chamber
42 Contaminant Particulates
44 Microscopic Particulates
46 Air Duct
48 Air Flow (Direction of Flow)
50 (UVGI) Treated Catalytic Fiber Optic Strand
52 (UVGI) Untreated Fiber Optic Strand
54 Catalyst Treatment (TiO₂ Coating)
56 Joined Fiber Sequence (A) (Weave pattern)
58 Joined Fiber Sequence (B) (Weave pattern)
60 Catalyzing Surface
62 Air Flow Aperture

The invention claimed is:

1. An apparatus for cleaning a contaminated fluid stream, comprising a prefilter, one or more photocatalytic chambers defined by said prefilter and one or more scrims further comprising a substrate and a photocatalytic coating adhered to the surface of said substrate, wherein said scrims define said photocatalytic chambers by being placed parallel to and apart from each other, and wherein each of said photocatalytic chambers further comprises an ultraviolet light source positioned within said photocatalytic chamber, and means for moving a fluid stream through said apparatus.

2. The apparatus of claim 1, wherein said fluid stream is a gas.

3. The apparatus of claim 2, wherein said photocatalytic coating further comprises a mixture of titanium dioxide and an organic polymer adhesive, wherein said mixture is coated on said substrate, and wherein said organic polymer adhesive is transparent or translucent to ultraviolet light.

4. The apparatus of claim 3, wherein said titanium dioxide comprises between 0.5 percent and 20 percent of the total weight of said scrim, and wherein said organic polymer adhesive comprises between 0.5 percent and 20 percent of the total weight of said scrim.

5. The apparatus of claim 3, wherein said titanium dioxide comprises between 2 percent and 5 percent of the total weight of said scrim, and wherein said organic polymer adhesive comprises between 2 percent and 5 percent of the total weight of said scrim.

6. The apparatus of claims 3, 4, or 5, wherein said substrate further comprises a woven fiberglass fabric.

7. A photocatalytic scrim comprising a substrate and a photocatalytic coating adhered to the surface of said substrate by an organic polymer adhesive.

8. The scrim of claim 7, wherein said photocatalytic coating further comprises a mixture of titanium dioxide and an organic polymer adhesive, wherein said mixture is coated on said substrate, and wherein said organic polymer adhesive is transparent or translucent to ultraviolet light.

9. The scrim of claim 8, wherein said titanium dioxide further comprises between 0.5 percent and 20 percent of the total weight of said scrim, and wherein said organic polymer adhesive further comprises between 0.5 percent and 20 percent of the total weight of said scrim.

10. The scrim of claim 8, wherein said titanium dioxide further comprises between 2 percent and 5 percent of the total weight of said scrim, and wherein said organic polymer adhesive further comprises between 2 percent and 5 percent of the total weight of said scrim.

11. The scrim of claims 8, 9, or 10, wherein said substrate further comprises a woven fiberglass fabric.
